(19) United States
(12) Patent Application Publication
(10) Pub. No.: US 2023/0138752 A1
(43) Pub. Date: May 4, 2023

(54) PLASTICIZERS TO IMPROVE RELEASE PERFORMANCE OF AMORPHOUS SOLID DISPERSIONS

(71) Applicant: Purdue Research Foundation, West Lafayette, IN (US)

(72) Inventors: Lynne Taylor, West Lafayette, IN (US); Clara Correa Soto, West Lafayette, IN (US)

(21) Appl. No.: 17/972,972

(22) Filed: Oct. 25, 2022

Related U.S. Application Data
(60) Provisional application No. 63/275,199, filed on Nov. 3, 2021.

(51) Int. Cl. A61K 9/20; A61K 31/4418; A61K 31/439

(52) U.S. Cl. CPC A61K 9/2077; A61K 9/2018; A61K 9/2027; A61K 9/2031; A61K 31/4418; A61K 31/439

(57) ABSTRACT

The invention generally relates to plasticizers to improve release performance of amorphous solid dispersions. In certain aspects, the invention provides an amorphous solid dispersion (ASD) composition including a polymer, a high glass transition (T_{g}) active pharmaceutical agent (API), and a plasticizer.

### RELATED APPLICATION

The present application claims the benefit of and priority to U.S. provisional patent application Ser. No. 63/275,199, filed Nov. 3, 2021, the content of which is incorporated by reference herein in its entirety.

### FIELD OF THE INVENTION

The invention generally relates to plasticizers to improve release performance of amorphous solid dispersions.

### BACKGROUND

Amorphous solid dispersion (ASD) is one of the most successful strategies to address the poor aqueous solubility of many new molecular entities. With more than 24 approved products on the market, whereby more than half of these have been approved in the past 14 years, ASDs are increasingly being employed to generate supersaturated solutions and improve oral bioavailability. Supersaturated solutions improve the membrane transport rate and, when permeability is not the limiting step, can facilitate adequate preclinical and clinical exposure. Amphiphilic polymers are an integral part of ASD formulations, serving multiple roles, in particular to delay active pharmaceutical (API) crystallization, and to increase the dissolution rate.

Although ASD is an attractive approach for delivery of poorly soluble APIs, notable challenges still remain. For instance, crystallization of the amorphous API is a major risk, and is detrimental to formulation performance due to a lost solubility advantage. In addition, although the amorphous API has a higher solubility than the crystalline counterpart, its dissolution rate is still slow. Much slower in fact when compared to the polymers typically used in the ASD formulation.

Consequently, a desirable attribute for ASDs is that the release of the components is controlled by the polymer, such that the API amorphous solubility is exceeded with the subsequent generation of drug-rich nanodroplets. It has been reported that the release of components from an ASD can be controlled by either the API or the polymer depending on the drug:polymer ratio. At low drug:polymer ratios, the ASD dissolves rapidly and completely under non-sink condition, and in this regime, the polymer controls the release of both drug and polymer. When the drug:polymer ratio increases, the release rate is slower and the drug concentration in solution does not usually exceed the amorphous solubility. Consequently, no drug-rich nanodroplets are formed. The maximum drug loading at which the polymer controls the release where complete and congruent release of API and polymer is obtained has been termed the limit of congruency LoC.

### SUMMARY

While the LoC has been found to vary considerably for different compounds, it is believed that the polymer-controlled dissolution is usually achieved only at a low drug:polymer ratio, in particular for high T_{g} compounds. This may impose constraints on the drug loading, increasing the amount of excipient in the formulation and leading to larger dosage sizes or multiple dosage units. In turn, an increased tablet burden may impact patient compliance, or subpar performance of the formulation may result if the drug loading is elevated beyond this limit.⁶

A relationship between high T_{g} and low LoC has emerged recently based on release studies of copovidone (PVPVA)-based ASDs. For instance, the LoC of indomethacin-PVPVA ASDs was reported to be 10% DL while its lower T_{g} (1.7° C.) analog, indomethacin ester had a 2.5-fold higher LoC. Similarly, the LoC of the high T_{g} compounds, atazanavir and ledipasvir has been reported to be ˜5% DL. In contrast, for lower T_{g} compounds such as ritonavir and miconazole, the LoC was reported as 25% DL and for clopidogrel and loratadine, LoC as high as 40% DL have been achieved.

Formulation strategies to push drug loading in ASD formulations is an active area of investigation. Different approaches explored to date to improve ASD release performance include polymer combinations, API protonation, and addition of surfactants. Surfactants are commonly incorporated into ASD formulations to increase drug release and as plasticizers to aid processability. For spray drying, a plasticizer may be added to enhance the formation of spherical and smooth-surfaced microcapsules. In the case of hot melt extrusion (HME), a surfactant may act as a plasticizer, reducing the processing temperature. HME is an attractive technology for commercial production of ASD intermediates, however, one limitation is the required high thermal energy input. Thus, it may not be useful for polymer requiring high thermal processing temperatures and for thermolabile drugs. Nevertheless, the use of plasticizers may allow lower processing temperatures to be employed, increasing the number of drugs that can be processed by HME. Some commonly used plasticizers include triacetin, citrate esters, D-α-tocopheryl, and surfactants. Additionally, low T_{g} drugs have been reported to act as plasticizers for the polymer in melt extruded dosage forms.

In recent work, it was suggested that surfactants increase the LoC of an ASD containing a high T_{g} API via their plasticizing effects. Therefore, it was of interest to further explore putative links between plasticizers and LoC as a potential strategy to increase the DL of ASDs containing high T_{g} APIs.

In the context of HME, plasticizers are selected for their ability to lower processing temperatures and reduce melt viscosity. Some additional requirements for a good plasticizer include low volatility, temperature stability, and compatibility. The efficiency of a plasticizer refers to the amount of plasticizer required to achieve the desired effect. For instance, the depression of the T_{g} of a polymer at a given plasticizer concentration is often used to define its efficiency, since T_{g} is one of the most important methods to measure chain mobility.

The invention recognizes that plasticizers increase the LoC of ASDs containing high T_{g} compounds. To that end, glycerol derivatives and citrate plasticizers were evaluated in terms of their ability to increase the LoC of ASDs containing the high T_{g} compounds, atazanavir and ledipasvir. Ritonavir, a lower T_{g} compound, was used as a control. The polymer employed for the ASDs was polyvinyl pyrrolidone vinyl acetate (PVPVA). Citrate plasticizers were selected because they are widely used with different polymers, are nontoxic, and have been approved for different applications including medical plastics, personal care, and food contact. Glycerol derivatives were evaluated to expand the chemical space of the plasticizers evaluated. Release rates from ASDs as a function of drug loading (DL) were monitored using surfaced normalized dissolution and various characterization methods were applied to the various ASDs.

In certain aspects, the invention provides an amorphous solid dispersion (ASD) composition comprising: a polymer, a high glass transition (T_{g}) active pharmaceutical agent (API); and a plasticizer. In another aspect, the invention provides a method for improving release of a high glass transition (T_{g}) active pharmaceutical agent (API) from an amorphous solid dispersion (ASD) composition, the method comprising formulating the ASD composition comprising the T_{g} API and a polymer with a plasticizer.

In certain embodiments, the plasticizer is present at no more than 15% (e.g., between 5-10%) in the composition. In certain embodiments, the high T_{g} API is present between 5% and 30% in the composition. In certain embodiments, a ratio of high T_{g} API:plasticizer in the composition is from about 5:1 to about 10:1. In certain embodiments, the plasticizer is a glycerol or glycerol derivative. In certain embodiments, the plasticizer is a citrate or citrate derivative. In certain embodiments, the polymer is Polyvinylpyrrolidone/vinyl acetate (PVP/VA). In certain embodiments, the high T_{g} API is a protease inhibitor, such as atazanavir or ledipasvir. In certain embodiments, the composition is in a form of a tablet.

### BRIEF DESCRIPTION OF THE DRAWINGS

FIGS. 1A-B show molecular structure of active pharmaceutical ingredients (APIs): atazanavir, ritonavir, and ledipasvir; and the plasticizers: triethyl citrate (TEC), triethyl acetyl citrate (TEAC), tributyl citrate (TBC), and tributyl citrate (TBAC). All structures, logP, and clogP were obtained using ChemDraw Professional 19.0 software. T_{g} for API are experimental values.

FIG. 2 is a graph showing limit of congruency (drug loading, DL) by weight for ASDs containing 5% w/w of different glycerol derivative plasticizers.

FIG. 3 is a graph showing normalized release rate for ATZ-PVPVA ASD containing 5% w/w plasticizer. ATZ 10 DL+GTA and ATZ 25DL+GTB. Neat PVPVA is added for comparison. The horizontal dashed line corresponds to the normalized release rate of neat PVPVA. Error bars represent standard deviation, n=3.

FIGS. 4A-B are graphs showing dissolution profile for ATZ 20% DL (A-D) and 25% DL (E-H) with 5% w/w of citrate plasticizer.

FIG. 5 is a graph showing limit of congruency (ATZ drug loading -DL) for ASDs containing 5% w/w of different citric acid derivative plasticizers.

FIG. 6 panels A-B are graphing showing normalized release rate for 20% DL ATZ (panel A) and at the LoC DL (panel B) for ATZ-PVPVA ASDs containing 5% w/w citric acid (CitAcd) or citrate plasticizers: TEC, TEAC, TBC, or TBAC. The horizontal dashed line corresponds to the normalized release rate of neat PVPVA. Error bars represent standard deviation, n=3.

FIG. 7 is a graph showing limit of congruency (LED drug loading- DL) containing 10% w/w of different plasticizers.

FIG. 8 is a graph showing normalized release rate for LED-PVPVA ASDs at the LoC (% DL) and containing 10% w/w of a plasticizer: TEAC, TBC or GTB. Neat PVPVA is added for comparison. The horizontal dashed line corresponds to the normalized release rate of neat PVPVA. Error bars represent standard deviation, n=3.

FIG. 9 is a graph showing normalized release rate for neat PVPVA and containing 10% w/w of a plasticizer, GTB or TBC. Error bars represent standard deviation, n=3.

FIG. 10 panels A-D is a set of graphs showing dissolution profile for RTV-PVPVA ASD containing GTB (panels C, D) or TBC (panels A, B) at 30DL (panels A, C) and 35DL (panels B, D).

FIG. 11 is a graph showing increase of LoC for ATZ, LED, RTV produced by the addition of TBC and GTB.

FIG. 12 panels A-B are graphs showing Percent water uptake versus time (panel A) and derivative of percent mass change versus time (panel B) for PVPVA and PVPVA containing 10% w/w of GTB or TBC.

FIGS. 13A-B panels A-F are graphs showing percent water uptake versus time (panels A, C, E) and derivative of percent mass change versus time. (panels B,D, F) for ATZ ASD exposed at 95% RH/37° C., for binary ASDs at 0, 5, 10, 20, and 30% DL (panels A,B); ternary ASDs containing TBC at 10, 20, and 30% DL and PVPVA+TBC 10% w/w/ (panels C,D); and ternary ATZ 20% DL ternary ASD containing 5% w/w of plasticizers GTB, TBC, TEC, or TEAC.

### DETAILED DESCRIPTION

Plasticizers are commonly used in the preparation of amorphous solid dispersion (ASD) with the main goal of aiding processability; however, the impact of plasticizers on drug release has not been explored. Diverse plasticizers including glycerol and citrate derivatives were evaluated as additives to increase the drug loading where good drug release could be achieved from copovidone- (PVPVA) based dispersions, focusing on high glass transition (T_{g}) drugs. ASDs were prepared using the high T_{g} compounds, atazanavir (ATZ) and ledipasvir (LED), as model drugs. The presence of a plasticizer at 5% w/w for ATZ and 10% w/w for LED ASDs, led to improved drug release. For the ATZ ASD, in the absence of plasticizer, release was very poor for drug loadings of 10% and above. Good release was obtained for plasticized ASDs up to a drug loading of 25%. The corresponding improvement for LED was from 5 to 20% DL. Interestingly, for a low T_{g} compound, ritonavir, relatively smaller improvements in release as a function of drug loading were achieved through plasticizer incorporation. The use of plasticizers represents a potential new strategy to increase drug loading in ASDs for high T_{g} compounds, and may help improve a major limitation of ASD formulations, namely the high excipient burden.

ASD Performance in the Presence of a Plasticizer

Plasticizers are commonly added to polymers to improve their processability, flexibility, and elasticity. In the pharmaceutical industry, plasticizers are commonly used in ASD formulations, especially in HME to reduce the processability temperature. For instance, 20% TEC was used as plasticizer for itraconazole(ITZ):Eudragit and ITZ:HP-55 ASDs produced by HME. The addition of a plasticizer allowed for a lower extrusion temperature, preventing polymer degradation and reducing its molten viscosity. Other small molecules including some APIs have been investigated as polymer plasticizers. For example, ibuprofen was used as plasticizer for Eudragit RS 30D during coating of nonpareil beads. Ibuprofen not only reduced the T_{g} of the film but also produced a smoother film. Surfactants are also widely used as plasticizers to increase processability and in some cases solubility. D-α-tocopheryl polyethylene glycol 1000 succinate (TPGS), poloxamer 407, and poloxamer 188 showed miscibility with HPMCAS at plasticizer concentrations of at least 20%. Additionally, 15% w/w surfactant decreased the processability temperature for HPMCAS-ITZ and substantially increased the drug release. The combination of drug and surfactant plasticization in the latter case reduced HPMCAS viscosity decreasing the processability temperature by 40° C. compared to neat HPMCAS. Other surfactants including Tween®80, docusate sodium, Myrj-52, Pluronic-F68, and sodium lauryl sulfate (SLS), all caused a solvation/plasticization effect manifested as a reduction of: the melting temperature of the API, the T_{g} of the polymer, and the combined T_{g} of the ASD. Plasticizers such as triethyl citrate (TEC) have been used for HME. For instance, 32.5C T_{g} reduction of Eudragit was reported when 12% TEC was added allowing lower HME processing temperatures. Similarly, itraconazole (ITZ) ASDs were prepared by HME using enteric polymers HPMCAS LG or Eudragit® L100-55. Polymers were pre-plasticized using 10% TEC to facilitate the hot-melt extrusion process. Moreover, the ITZ-HPMCAS formulation using TEC and TPGS showed superior dissolution performance compared to other hydrophilic additives.

For instance, reduction of T_{g} of Eudragit® RS PO was reported to be 32.5° C. when 12% TEC was added allowing for a lower HME processing temperature. Similarly, itraconazole (ITZ) ASDs were prepared by HME using enteric polymers HPMCAS LG or Eudragit® L100-55. Polymers were pre-plasticized using 10% TEC to facilitate the hot-melt extrusion process. Moreover, the ITZ-HPMCAS formulation using TEC and TPGS showed superior dissolution performance compared to other hydrophilic additives.

In the present study, a plethora of plasticizers was evaluated to enhance release from ASDs, specifically to increase the LoC of high T_{g} compounds. All evaluated plasticizers produced a reduction in the ASD T_{g} compared to the binary ASD (Table 4).

TABLE 4
T_{g} of ASD and the T_{g} reduction produced by
the addition of 5% w/w of a plasticizer

| | | T_{g} reduction (° C.) compared to |
|---|---|---|
| ASD sample | T_{g} (° C.) | the binary ASD |
| PVPVA | 108.5 | |
| ATZ 5% DL (LoC) | 108.9 ± 0.2 | |
| ATZ 20% DL | 109.0 ± 0.1 | REF |
| ATZ20% DL + TEC | 91.5 ± 0.8 | 17.5 |
| ATZ20% DL + TEAC | 94.0 ± 0.3 | 14.9 |
| ATZ20% DL + TBC | 92.8 ± 0.1 | 16.2 |
| ATZ20% DL + TBAC | 93.7 ± 0.4 | 15.3 |
| ATZ20% DL + GTB | 89.3 | 19.7 |
| PVPVA + GTB | 73.0 ± 0.5 | 35.5 |
| PVPVA + TBC | 77.7 ± 1.6 | 30.8 |

T_{g} has been previously pointed out as an important API attribute that impacts LoC. High T_{g} APIs have been repeatedly reported to have a low LoC which may result in tablet burden or suboptimal drug concentration. This research suggests that the use of a plasticizer may increase the mobility of the ASD which promotes dissolution; therefore, they can be used to both improve processability and to increase the LoC.

Mechanism of Dissolution Enhancement by Plasticizers

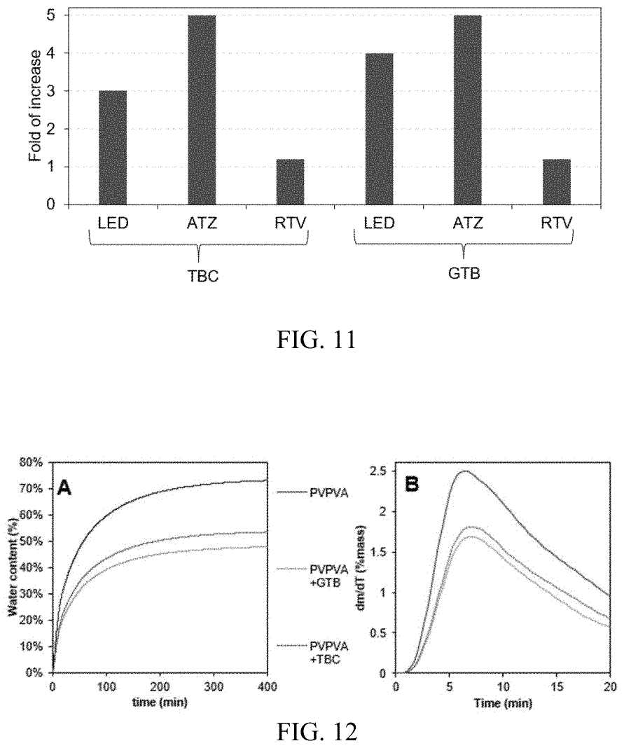

As previously mentioned, it is desirable to formulate an ASD at a drug:polymer ratio in which the dissolution is controlled by the polymer. Thus, it is relevant to consider the polymer dissolution process. Polymer dissolution is mainly controlled by three processes: solvent ingress, chain disengagement and polymer diffusion away from the interface. As the solvent enters the glassy polymer matrix, a gel is produced, and polymer chains start to disentangle in a reptation process with a formation of a gel-liquid interface between the rubbery polymer and the solvent. Thus the chains get solvated, disentangle, and reptate out of the gel phase. The transport of the polymer chains may be solvent penetration, disengagement or diffusion limited. For the case of a glassy high molecular weight polymer such as PVPVA, dissolution will most likely be controlled by disengagement of the chains. It has been demonstrated that the presence of small molecules may act as a plasticizer increasing the mobility of the chains in the gel phase with a consequent increase in the dissolution rate of the polymer. The results presented in FIG. 11 above support that polymer disentanglement is key rate process positively impacted by the presence of a plasticizer, given that the rate of water sorption into the polymer matrix is reduced by the presence of the plasticizers.

Dissolving, swelling, and plasticizing problems in polymers are very closely connected. A plasticizer improves the workability and flexibility of the polymer by increasing the intermolecular separation of the polymer molecules. Different theories have been proposed to explain plasticization of polymers including, the lubricity theory, the gel theory, and the free volume theory. The free volume theory gives a more precise explanation of plasticization. In general, plasticization theories concur that plasticizers act by penetrating into the polymer producing a separation of the polymer chains and reducing the intermolecular forces between the polymer chains.

The free volume theory rationalized the T_{g} reduction by plasticizers.⁴² Glass transition temperature marks the transition from a glassy to rubbery solid. The viscosity for all polymers at the T_{g} is around 10¹² Pa·s, regardless of the polymer chemical structure, and thus friction between molecules, viscosity, is releated to the volume and the T_{g} . In other words, at the T_{g}, all material exhibit the same fractional free volume.

Since penetration of the plasticizer into the polymer and therefore interaction between the two components is crucial, it is necessary that neither the plasticizer nor the polymer tend to preferentially self-associate at the expense of polymer-plasticizer interactions. For the case of PVPVA, no intramolecular hydrogen bonding (HB) is expected, while for glycerol, self-association via hydrogen bonding is possible, and this may contribute to the poor performance of glycerol at increasing the dissolution performance for ATZ-PVPVA ASDs. Similar finding was previously reported in which no plasticization of PVC was found by glycerol and this was attributed to the strong glycerol-glycerol intermolecular interactions that prevent PVC-glycerol interactions. Moreover, the rate of diffusion of the plasticizer in the polymeric matrix is one of the main factors that determine the plasticizer efficiency and depends on the size and molecular weight of the plasticizer. Usually, smaller molecules have higher diffusion rates; nevertheless, the smaller the molecule the greater the volatility and higher the risk of lost from the plasticized product. For instance, citrate plasticizers TEC, TBC, TEAC and TBAC were evaluated as plasticizers for poly lactic acid (PLA). Films were prepared by extrusion at 130° C. to 160° C., and a reduction in plasticizer concentration due to evaporative loss, especially for the lower molecular weight (TEC), was evident. The extent of loss was linear with the plasticizer content and molecular weight. Likewise, the internal mobility of the plasticizer is important for a plasticizing effect and in general, the T_{g} reduction will be proportional to the temperature difference between the polymer T_{g} and the plasticizer T_{g}.

The glycerol and glycerol derivative plasticizers evaluated in this research have a molecular weight range from 92 to 302 g/mol whereby glycerol is the lowest molecular weight plasticizer and GTB is the largest. The probability of intermolecular hydrogen bonding for glycerol and its low molecular weight may be the reasons why it performs poorly with only a 1.5 increase in the LoC of ATZ while GTB produced a 5-fold increase in the LoC. For citrate derivatives, the molecular weight ranges from 276 g/mol for TEC to 402.5 g/mol for TBAC where only TEC contains an OH group capable of forming HB. Consequently, TEC was the least efficient of the evaluated citrate at increasing the LoC of ATZ-PVPVA ASD.

The dissolution rate of all the ASD below the LoC containing plasticizers were higher than that of the neat polymer. Moreover, the dissolution rate of the polymer containing GTB or TBC was as much as twice that of the neat polymer with a T_{g} reduction of up to 35° C. Conversely, the water sorption of the polymer was reduced in the presence of GTB or TBC. Combining the polymer dissolution and the plasticizer theory, it is reasonable to suggest that the main mechanism by which plasticizers enhance dissolution rate and consequently LoC of the high T_{g} API is by increasing the mobility of the polymer chains. In other words, the disentanglement rate is enhanced by the presence of a plasticizer. Some authors have suggested that plasticizers may affect the water uptake especially for surfactants which show plasticizer effects and that increase wettability; however, for the plasticizers evaluated herein the opposite was observed which is not surprising due to the relatively high lipophilicity of the evaluated plasticizers. Withou being limited by any particular theory or mechanism of action, it is believed that small molecules such as residual solvent increases the dissolution rate due to diffusional properties that enhance polymer chain mobility and promote disentanglement.

In this application we provide an approach to overcome one of the main challenges faced by ASD formulations namely the low LoC experienced for some APIs. Using plasticizers as an additive in the formulation may increase the LoC especially for high T_{g} APIs which is beneficial for maintaining a small dosage form size.

### CONCLUSION

Several plasticizers including glycerol and citrate derivatives were evaluated as additives for ASD formulations containing high T_{g} drugs to increase the limit of congruency. 5% w/w of citrate plasticizers and the most lipophilic glycerol derivative evaluated, GTB, increased the LoC of ATZ-PVPVA ASDs by up to 5-fold. For the higher T_{g} compound, LED, 10% w/w plasticizer was necessary to produce a 4-fold increase in the LoC. In all cases, the dissolution rate was higher than that of the neat polymer, for drug loadings below the LoC. The suggested mechanism by which the plasticizers increase the dissolution performance at a higher DL is through enhancing the polymer disentanglement rate. Addition of plasticizers will be useful to expand the use of HME for ASD processing. The observations from the current study provide a strategy for formulators to enhance the drug loading in ASDs without compromising their release performance.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Materials

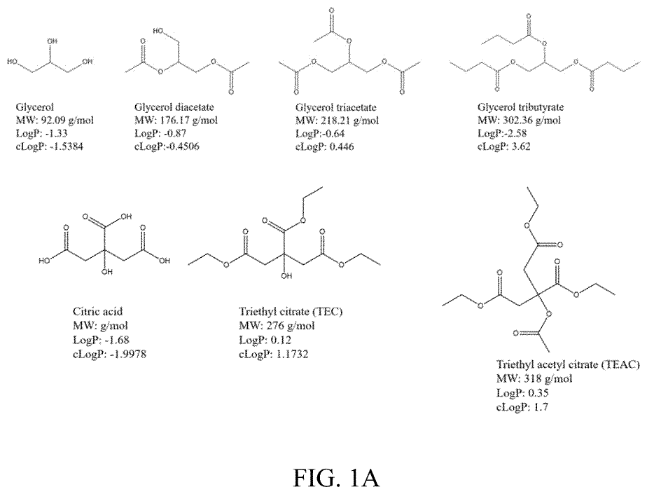

Atazanavir free base, and ritonavir were purchased from ChemShuttle (Wuxi City, China), while ledipasvir was purchased from Gojira Fine Chemicals (Bedford Heights, Ohio, USA). Polyvinyl pyrrolidone vinyl acetate 64 copolymer (PVPVA) was obtained from BASF (Ludwigshaven, Germany). Glycerol and citric acid were purchased from Fisher Scientific (Hampton, N.H., USA). Glycerol triacetate (GTA), Tri-n-butyl citrate (TBC), and triethyl citrate (TEC) were obtained from Alfa Aesar (Haverhill, Mass., USA). Tributyl-o-acetylcitrate (TBAC), triethyl-o- acetylcitrate (TEAC), and glycerol diacetate (diacetin) were purchased from TCI America, Inc. (Portland, OR, USA). Glycerol tributyrate (GTB) was obtained from Sigma Aldrich (St. Louis, Mo., USA). Structures shown in FIGS. 1A-B.

All dissolution experiments were conducted in 50 mM phosphate buffer pH 6.8 prepared by dissolving 3.52 g of sodium phosphate monobasic monohydrate and 3.48 g of sodium phosphate dibasic anhydrous to 1L with Milli-Q water. The pH was adjusted to pH 6.8 if needed using HCl or NaOH.

### Example 2: ASD Preparation

Solvent evaporation was used to prepare the ASDs. API (ATZ, RTV, or LED), PVPVA, and plasticizer were dissolved in sufficient methanol and sonicated until a clear solution was obtained. The solvent was removed using a Heidolph rotary evaporator (Schawabach, Germany). The water bath was set at 55° C. and the rotation speed at 170 rpm. After the solvent was removed, the ASDs were further dried under vacuum at room temperature for at least 24 h. After drying, the glassy solid was powdered using a mortar and pestle and sieved through a 250 μm sieve. All the ASDs were confirmed amorphous by the absence of sharp peaks in the powder X-ray diffraction pattern (PXRD).

The amount of plasticizer added to the ASDs was 5 g per 100 g of API and polymer for ATZ and RTV, whereas 10 g of plasticizer was added per 100 g of LED and PVPVA. For simplicity, drug loadings are expressed as the weight ratio of drug. For instance, atazanavir 10% drug loading (ATZ 10% DL) refers to 10 g of ATZ and 90 g of PVPVA for the binary ASD, and to 10 g of ATZ, 90 g of PVPVA, 5 g of plasticizer for the ternary ASDs. And in the case of LED, LED 10% DL will refer to 10 g of LED, 90 g of PVPVA, and 10 g of plasticizer.

### Example 3: Surface Normalized Dissolution

Surface normalized dissolution was employed to evaluate the release performance of the ASDs and to calculate the normalized release rate. A Hanson Vision G2 dissolution tester with a Vision heater (Teledyne Hanson Chatsworth, CA) was utilized. A tablet was prepared immediately prior to the dissolution test by compressing 100 mg of ASD in an 8 mm die at 2 500 psi for 60 seconds yielding a tablet surface are of 50 mm². The temperature was maintained at 37° C., the rotation speed was 100 rpm, and the dissolution medium was 50 mM phosphate buffer pH 6.8 degassed and pre-equilibrated at 37° C. 100 to 200 μl of sample was withdrawn every 5 min for the initial 20 minutes, then every 10 minutes until 60 minutes and finally at 75, 90 and 120 minutes. The medium was replenished after each sample withdrawal to maintain a constant volume.

The concentration of API and polymer during the dissolution was assessed by high performance liquid chromatography (HPLC) using an Agilent 1100 system (Agilent Technologies, Santa Clara, Calif.). An Ascentis® Express C18 10 cm×3 mm with 2.7 μm particle size column (Sigma-Aldrich St. Louis, Mo.) was employed for separation of ATZ, RTV, and LED. The column used for PVPVA was a size exclusion chromatography (SEC), 300 mm×8 mm A 2500 Viscotek with an exclusion limit of 10 000 Da for pullulan (Malvern Panalytical, Malvern, UK). For ATZ and LED, the mobile phase was aqueous TFA 0.1% v/v:acetonitrile (ACN) 6:4 v/v at a flow rate of 0.5 ml/min, injection volume 10 detection wavelength (X.) 210 nm, and column temperature 40° C. For RTV, the mobile phase was aqueous TFA 0.1% v/v:ACN 6:4 v/v, flow rate 0.5 ml/min, injection volume 10 detection λ 241 nm and column temperature 40° C. For PVPVA, the mobile phase was water:methanol 3:7 v/v at a flow rate of 0.5 ml/min, injection volume 50 detection λ 205 nm, and column temperature 37° C. A linear calibration curve (r² 0.999 obtained for all cases) was built for each of the analytes in the range 0.5 to 30 μg/ml for ATZ, 1 to 40 μg/ml for RTV, 1 to 40 μg/ml for LED, and 5 to 80 μg/ml for PVPVA.

Nanodroplets size was measured after 90 minutes dissolution using dynamic light scattering (DLS) performed with a Zetasizer Nano ZS (Malvern Instruments, Westborough, MA). The instrument was set to backscatter mode at an angle of 173° and the temperature holder was set to 37° C.

The surface normalized dissolution rate (R) was calculated based on the mg released versus time profile. The slope from cumulative mg release vs time represents the release rate (k). To normalize, k was divided by the tablet surface area (S, 0.5 cm²) times the fraction of the component (x) (Equation 1)

$$R = \frac{k}{s \times x} \qquad Equation 1$$

### Example 4: Glass Transition (T_{g}) and Miscibility Determination by Differential Scanning Calorimetry (DSC)

Glass transition temperature and plasticizer-API miscibility was determined using a TA Q2000 differential scanning calorimeter (DSC) (TA Instruments, New Castle, DE) with a refrigerated cooling accessory (RCS) using N₂ as the purge gas at a flow of 50 ml/min. For T_{g} determination, around 5 mg of sample was accurately weighed into an aluminum pan with a Tzero lid and subject to a cool-heat-cool cycle. Samples were cooled at -20° C. and heated up to 220° C. The heating was done in modulated mode at a heating rate of 2° C/min with a modulation period of +/−1° C. every 60 seconds. The midpoint temperature of the heat capacity change in the thermogram from the second heating cycle scan was taken as the T_{g}. The DSC was calibrated with indium at the appropriate heating rate.

API:plasticizer at a ratio 8:1 was prepared by rotary evaporation to evaluate miscibility and T_{g} depression produced by the plasticizer. 3 to 5 mg was accurately weighed in an aluminum pan with a hermetic lid with a pinhole. Since the T_{g} was evaluated after only 1 hour of vacuum drying to avoid crystallization, a second drying was done by employing an isothermal hold at 20° C. in the DSC with nitrogen flow for at least 20 minutes. Then, the sample was cooled to −60 and heated at 2° C./min to 210° C. with a modulation period +/−1° C. every 60 seconds.

### Example 5: Dynamic Vapor Sorption (DVS)

The isothermal water sorption at 37° C. was measured for selected ASDs using a DVS-Advantage device (Surface Measurement System Ltd Allentown, Pa.). Around 5 mg of sample was first dried at 0% relative humidity (RH) and 37° C. for one hour followed by exposure to 95% RH and 37° C. for at least 8 hours. Percent of water sorbed and derivative of percent mass over time (dm/dT %) was analyzed.

### Example 6: Results

Surface Normalized Dissolution

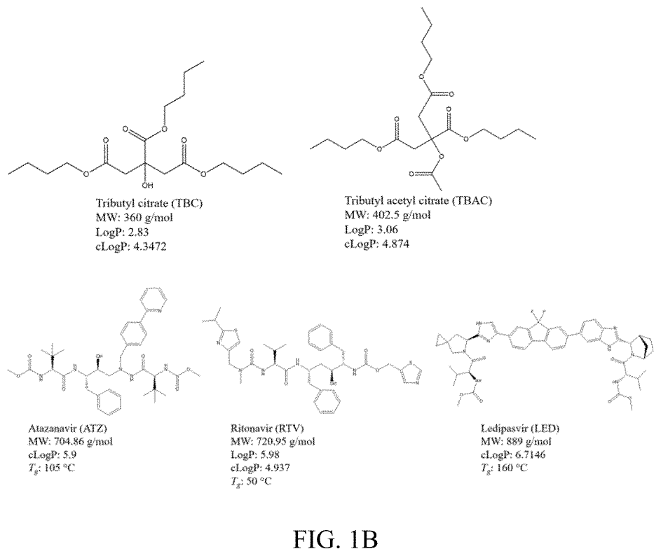

Glycerol and glycerol derivatives: glycerol diacetate (GDA), glycerol triacetate (GTA) and glycerol tributyrate (GTB) were evaluated for their ability to increase the limit of congruency (LoC) for ATZ-PVPVA ASDs, namely the maximum drug loading at which a complete and congruent release of API and polymer is obtained. We previously reported the LoC for the binary ATZ-PVPVA ASD to be 5% DL while at 10% DL minimum release of ATZ is produced (Chapter 3). For ASDs containing 5% w/w of glycerol derivatives plasticizers, the LoC was increased to 7.5% DL in the case of glycerol and GDA, to 10% DL in the case of GTA, and to 25% DL for GTB as is shown in FIG. 2.

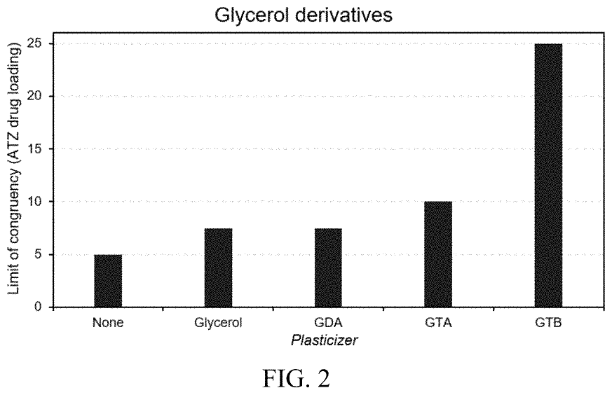

GTA and GTB not only increased the LoC but also the normalized release rate of both drug and polymer whereby the normalized release was faster than that of neat PVPVA, as shown in FIG. 3.

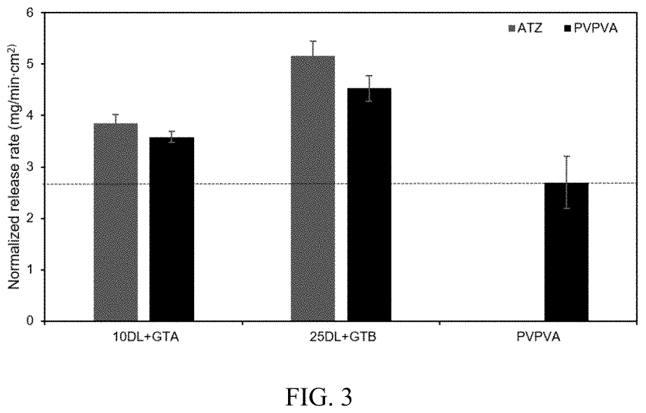

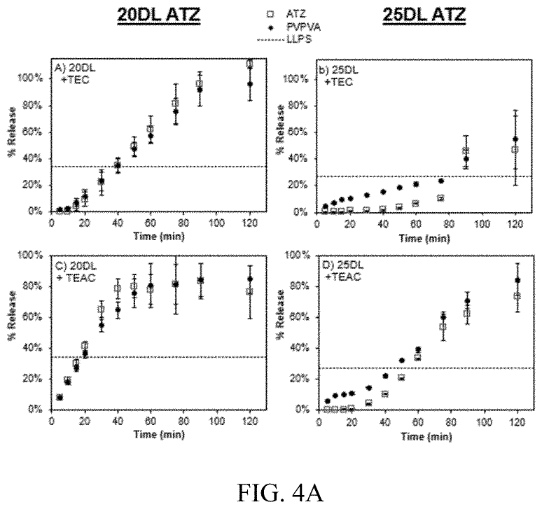

The LoC of ATZ-PVPVA ASDs containing 5% w/w citric acid or citrate derivative plasticizers TEC, TEAC, TBC, and TBAC was evaluated. FIGS. 4A-B present the release profiles for ATZ 20% DL and 25% DL in the presence of the citrate plasticizers. Citric acid slightly increased the LoC from 5 to 7.5% DL. In contrast, TEC increased the LoC to 20% DL while the LoC for ASDs containing TEAC, TBC and TBAC was 25% DL (FIG. 5). In other words, incorporation of 5% w/w of a citrate derivative plasticizer can increase the LoC of ATZ up to 5-fold with respect to the binary ASD (LoC=5% DL). In the case of TEC, TEAC, and TBAC, a lag time of around 20 min was observed in the release profile at the LoC, 20% DL for TEC and 25% DL for TEAC and TBAC, (FIG. 4) followed by almost complete release of drug and polymer. The lag time was not observed for ATZ 20% DL+TEAC but was evident when the DL was increased to 25%. When a lag time was observed, the normalized release rate was calculated from the linear region of the release profile, after the lag time (from 30 to 60 min). In all cases, the LLPS concentration was reached, with the subsequent formation of drug-rich nanodroplets. The size of the nanodroplets varies from 500 to 1500 nm Z-average as measured by DLS.

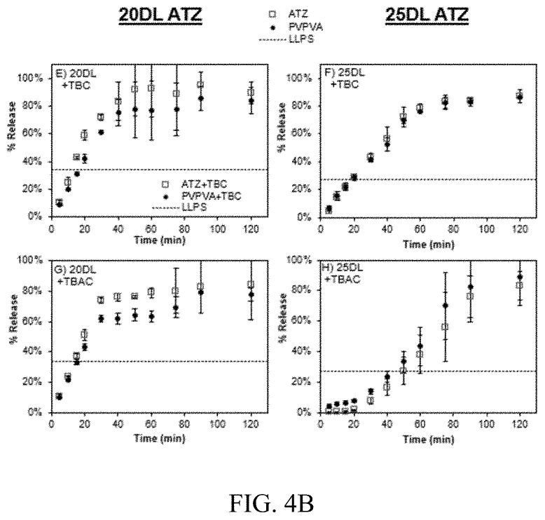

At 20% DL, complete release of drug and polymer was observed for all ATZ ternary ASDs containing citrate derivatives. Additionally, the normalized release rate was higher compared to neat polymer for all citrate plasticizers with the exception of TEC (FIG. 6 panel A);

whereas at the limit of congruency, the release rate was similar to that of the neat polymer. (FIG. 6 panel B).

Some of the plasticizers that were effective at increasing the LoC of ATZ were evaluated for the high T_{g} compound ledipasvir (LED). LED has a dry T_{g} of 160° C. and a LoC for the binary LED-PVPVA ASD of 5% DL. If the low mobility, reflected in a high T_{g}, is the limitation for drug release, it is expected that the addition of a plasticizer would increase the LoC. Citrate plasticizers and the glycerol derivative GTB were studied at 10% DL LED with 5% w/w of plasticizer. Only citric acid resulted in complete release while minimum release was obtained for the other plasticizers (data not shown). Since the T_{g} of LED is around 55° C. higher than ATZ, it was rationalized that additional plasticizer is required. Thus, the amount of plasticizer was increased to 10% w/w to produce a higher reduction in the T_{g} of the LED ASDs. Table 1 shows that an increase in plasticizer amount from 5 to 10% w/w for LED 10% DL reduced the ASD T_{g} by 10° C. with a subsequent increase in total release from 12 to 20% in the case of TEC, while for TEAC, the ASD T_{g} was reduced by 14° C. and the total release increased from 20% to complete release.

TABLE 1
T_{g} of ternary LED ASD containing
5% w/w or 10% w/w of TEC or TEAC.

| Sample | T_{g} (° C.) | % LED released by 90 min |
|---|---|---|
| LED 10% DL + TEC 5% | 95.6 | 12% |
| LED 10% DL + TEC 10% | 84.9 | 20% |
| LED 10% DL + TEAC 5% | 99.1 | 20% |
| LED 10% DL + TEAC 10% | 85.8 | 94% |

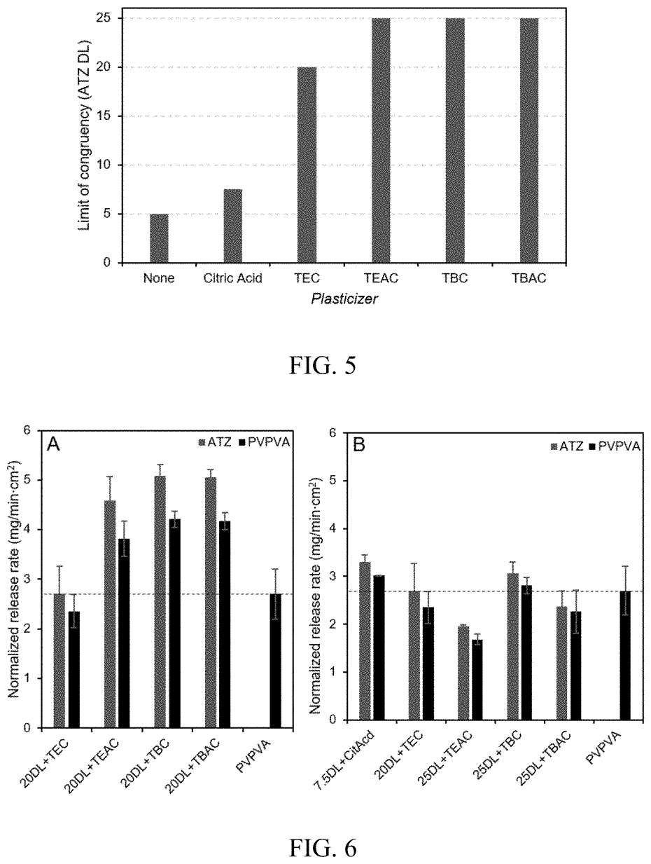

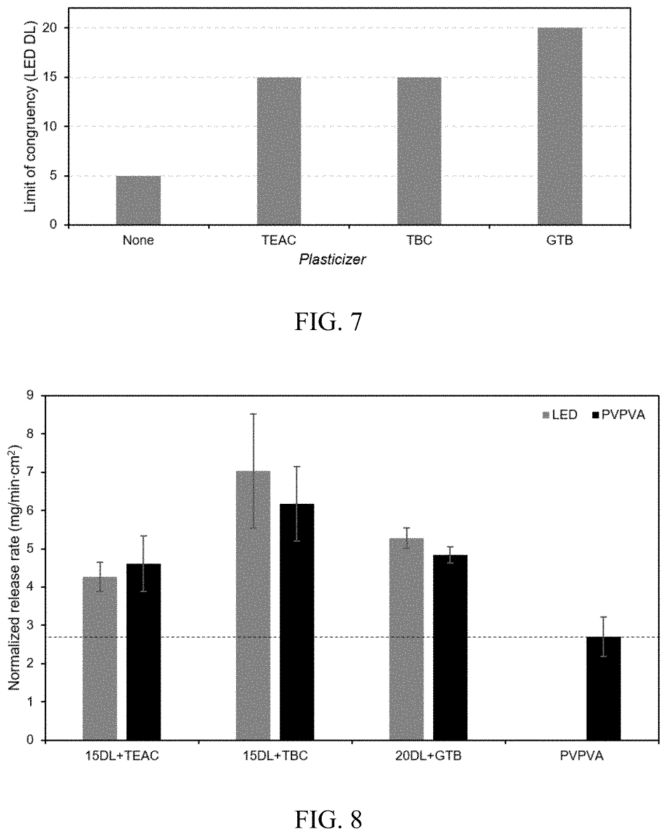

At the higher plasticizer content and for 10% DL LED ASDs, complete drug release was achieved with TEAC, TBC, and GTB. On the other hand, less than 30% of the drug was released for ASDs containing TEC or TBAC. TEAC, TBC and GTB were selected for further studies. The LoC of LED was increased from 5 to 15% DL with TEAC or TBC, and to 20% DL with GTB (FIG. 7). The normalized release rate for these ASDs at the LoC were higher than that of the neat PVPVA as shown in FIG. 8.

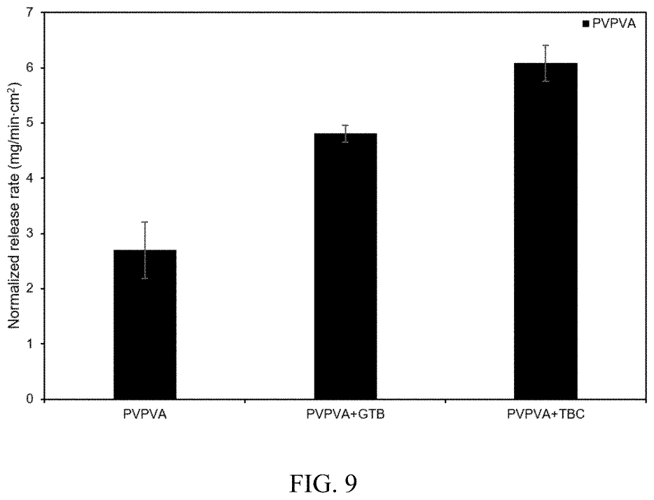

As a control, the dissolution of PVPVA containing 10% w/w of GTB or TBC was evaluated, and the normalized release rates are presented in FIG. 9. Both plasticizers produced a faster dissolution rate than that observed for neat PVPVA.

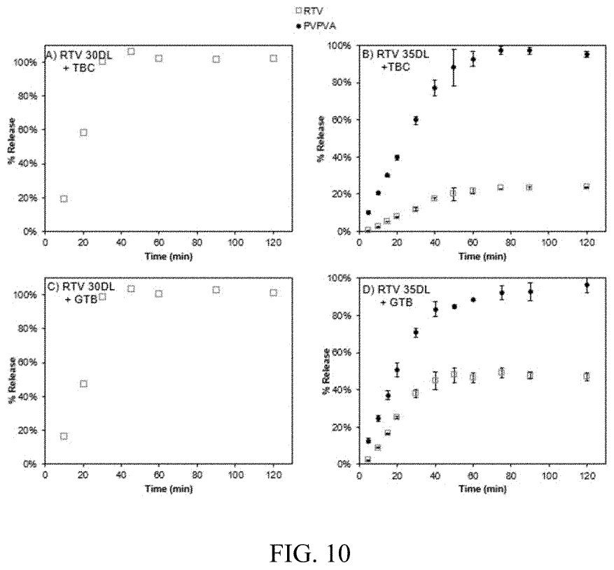

A possible reason for the increase in the LoC for the two high T_{g} compounds is plasticization, which increases the mobility, as reflected in a lower T_{g}. Therefore, it is expected that little improvement will be obtained for a lower T_{g} compound. To evaluate this hypothesis, an ATZ analog, ritonavir (RTV) was evaluated with TBC and GTB. RTV has a T_{g} of 50° C. and the RTV-PVPVA ASD has a LoC of 25% DL therefore mobility is expected to be less of a limitation. 5% w/w/ of GTB or TBC was added to 30% DL and 35% DL RTV-PVPVA ASDs. Complete release was observed at 30% DL but not at 35% DL (FIG. 10 panels A-D).

FIG. 11 shows the extent of the increase in LoC for ATZ, LED, and RTV with the plasticizers GTB and TBC. ATZ LoC was 5-fold higher than the binary ASD upon adding TBC or GTB, while for LED, the LoC was 3-fold and 4-fold higher with TBC and GTB respectively. On the other hand, only a 1.2-fold increase in the LoC was obtained in the case of RTV.

Tg Measured by Differential Scanning Calorimeter (DSC)

DSC was used to evaluate the API:plasticizer miscibility and plasticizing effect. All plasticizers produced a notable reduction in the T_{g} of ATZ and LED. The citrate plasticizers produced a similar extent of T_{g} depression of around 30° C. (Table 2) for ATZ and 50° C. for LED (Table 3).

TABLE 2
ATZ:Plasticizer 8:1 amorphous mix T_{g} and
T_{g} reduction compared to neat amorphous ATZ.

| Sample | T_{g} (° C.) | T_{g} reduction (° C.) |
|---|---|---|
| ATZ | 105.5 | N/A |
| ATZ:glycerol 8:1 | 72.8 ± 0.9 | 32.7 |
| ATZ:GTA 8:1 | 62.2 ± 2.3 | 43.3 |
| ATZ:TEC 8:1 | 71.5 ± 0.8 | 34.0 |
| ATZ:TEAC 8:1 | 75.1 ± 1.2 | 30.4 |
| ATZ:TBC 8:1 | 72.1 ± 0.4 | 33.3 |
| ATZ:TBAC 8:1 | 74.6 ± 0.4 | 30.9 |

TABLE 3
LED:Plasticizer 8:1 amorphous mix T_{g} and
T_{g} reduction compared to neat amorphous LED.

| Sample | T_{g} (° C.) | T_{g} reduction (° C.) |
|---|---|---|
| LED | 162 | — |
| LED:TEC 8:1 | 109.1 ± 2.0 | 53.1 |
| LED:ATEC 8:1 | 114.6 ± 2.0 | 47.6 |
| LED:TBC 8:1 | 111.0 ± 1.4 | 51.1 |
| LED:ATBC 8:1 | 113.2 ± 1.1 | 48.9 |

Dynamic vapor sorption (DVS) of ASDs

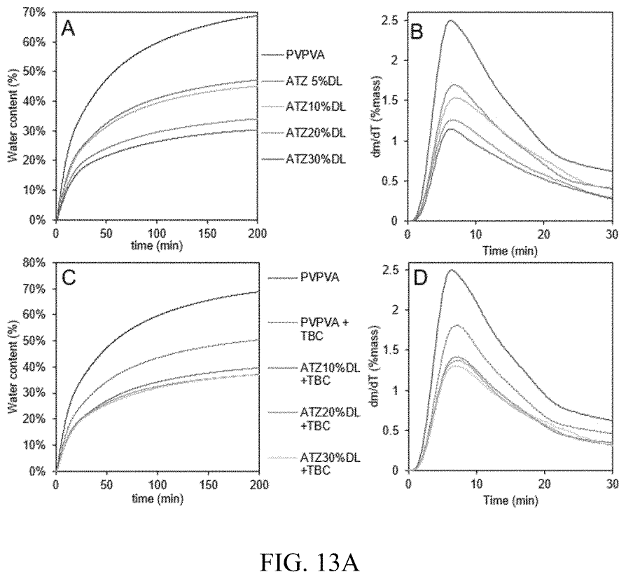

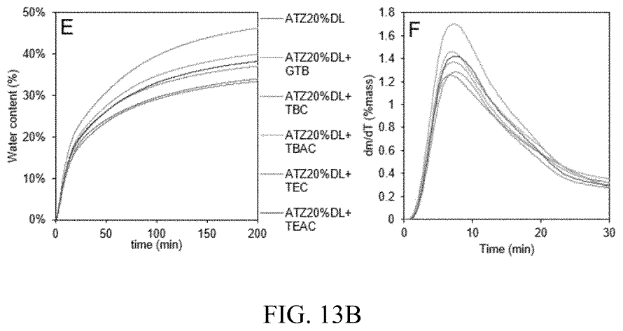

Water sorption kinetics of select samples were investigated to evaluate the impact of plasticizers on the water uptake. FIG. 12 panels A-B show the percent water sorbed versus time for ATZ-PVPVA binary ASD at 0, 5, 10, 20, and 30% DL. It is clear that as the DL increases, the percent water uptake decreases. This follows from the high lipophilicity of ATZ. The rate of water uptake, i.e. how fast the water sorption occurs until a plateau is reached, can be seen from a plot of the derivative of percent mass change over time (dm/dT %/min). The maximum of the dm/dT plot decreases as the DL increases and the maximum rate is observed in about 7 minutes. The maximum water content follows the same trend as the kinetics, in which the total percent of water sorbed decreases as the DL increases. The decrease in total water content is more prominent when the DL changes from 10 to 20% relative to from 20 to 30% in the case of binary ASDs. For the ternary ASD containing 5% w/w TBC the water uptake is similar for 10, 20 and 30% DL (FIG. 13A panels C-D).

The water content for the 20% DL ATZ ternary ASD containing 5% w/w of GTB, TBC, TEC, TEAC, or TBAC was evaluated and compared with the binary 20% DL ATZ and the results are presented in FIG. 13B panels E-F. TEC present a very similar water uptake as the binary ASD. Comparatively, only a slight increase in water uptake was observed for TBC, TEAC and a more prominent increase of water uptake was found for the ASD with GTB. PVPVA is a hydrophilic polymer and sorbed a considerable amount of water (80% water content). The presence of plasticizers such as TBC or GTB dramatically reduced the polymer water sorption (FIGS. 13A-B panels A-F). The reduction is expected due to the high lipophilicity of these plasticizers (Log P around 2.5).

What is claimed is:

1. An amorphous solid dispersion (ASD) composition comprising:
a polymer
a high glass transition (T_{g}) active pharmaceutical agent (API); and
a plasticizer.

2. The ASD composition of claim 1, wherein the plasticizer is present at no more than 15% in the composition.

3. The ASD composition of claim 1, wherein the high T_{g} API between 5% and 30% in the composition.

4. The ASD composition of claim 1, wherein a ratio of high T_{g} API:plasticizer in the composition is from about 5:1 to about 10:1.

5. The ASD composition of claim 1, wherein the plasticizer is a glycerol or glycerol derivative.

6. The ASD composition of claim 1, wherein the plasticizer is a citrate or citrate derivative.

7. The ASD composition of claim 1, wherein the polymer is Polyvinylpyrrolidone/vinyl acetate (PVP/VA).

8. The ASD composition of claim 1, wherein the high T_{g} API is a protease inhibitor.

9. The ASD composition of claim 8, wherein the protease inhibitor is atazanavir or ledipasvir.

10. The ASD composition of claim 1, wherein the composition is in a form of a tablet.

11. A method for improving release of a high glass transition (T_{g}) active pharmaceutical agent (API) from an amorphous solid dispersion (ASD) composition, the method comprising formulating the ASD composition comprising the T_{g} API and a polymer with a plasticizer.

12. The method of claim 11, wherein the plasticizer is present at no more than 15% in the composition.

13. The method of claim 11, wherein the high T_{g} API between 5% and 30% in the composition.

14. The method of claim 11, wherein a ratio of high T_{g} API:plasticizer in the composition is from about 5:1 to about 10:1.

15. The method of claim 11, wherein the plasticizer is a glycerol or glycerol derivative.

16. The method of claim 11, wherein the plasticizer is a citrate or citrate derivative.

17. The method of claim 11, wherein the polymer is Polyvinylpyrrolidone/vinyl acetate (PVP/VA).

18. The method of claim 11, wherein the high T_{g} API is a protease inhibitor.

19. The method of claim 18, wherein the protease inhibitor is atazanavir or ledipasvir.

20. The method of claim 11, wherein the composition is in a form of a tablet.
